(19) United States
(12) Patent Application Publication
(10) Pub. No.: US 2007/0298099 A1
(43) Pub. Date: Dec. 27, 2007

(54) Liquid and Semi-Solid Pharmaceutical Formulations for Oral Administration of a Substituted Amide

(76) Inventors: Andrey V. Peresypkin, Cranford, NJ (US); Eleni Dokou, Harleysville, PA (US); Craig McKelvey, Ambler, PA (US); Charles DeLuca, Royersford, PA (US); Laman L. Alani, Lansdale, PA (US); Todd Gibson, North Haven, CT (US); Danielle H. Euler, Souderton, PA (US); Santipharp Panmai, West Point, PA (US); W. Peter Wuelfing, Schwenksville, PA (US); Thomas P. Gandek, Collegeville, PA (US); Drazen Ostovic, Newbury Park, CA (US); Timothy Rhodes, South Orange, NJ (US); Brian K. Hamilton, Lansdale, PA (US)

Correspondence Address: MERCK AND CO., INC, P O BOX 2000, RAHWAY, NJ, 07065-0907, US

(21) Appl. No.: 11/667,344

(22) PCT Filed: Nov. 18, 2005
(86) PCT No.: PCT/US05/41836

§ 371 Date: May 8, 2007

Related U.S. Application Data
(60) Provisional application No. 60/630,746, filed on Nov. 24, 2004.

(51) Int. Cl. A61K 9/48; A61K 31/44; A61P 25/30; A61P 3/04; A61P 3/10

(52) U.S. Cl. 424/456; 514/351

(57) ABSTRACT

N-[1S,2S]-3-(4-chlorophenyl)-2-(3-cyanophenyl)-1-methylpropyl]-2-methyl-2-{[5-trifluoromethyl]pyridine-2-yl}oxy}propanamide (Compound I) has surprisingly improved solubility and bioavailability in a lipophilic vehicle comprising a pharmaceutically acceptable digestible oil, a surfactant, or a cosolvent, or a mixture of any two or more thereof. In one embodiment of the present invention are self-emulsifying or self-microemulsifying composition comprising 1) Compound I; 2) a surfactant having an HLB of 1 to 8; and 3) a surfactant having an HLB of over 8 to 20; and optionally, 4) a digestible oil and/or cosolvent and/or antioxidant or preservative.

### BACKGROUND OF THE INVENTION

The compound N-[1S,2S]-3-[(4-chlorophenyl)-2-(3-cyanophenyl)-1-methylpropyl]-2-methyl-2-{[(5-trifluoromethyl)pyridine-2-yl]oxy}propanamide (Compound I), described in WO 03/077847, is a cannabinoid 1 (CB 1) receptor modulator, more particularly a functional CB 1 antagonist, and even more particularly, a CB 1 inverse agonist. This invention relates to formulations of Compound I and pharmaceutically acceptable salts and solvates thereof for use in mammals, especially humans, especially encapsulated formulations, including hard and soft gelatin capsules, which formulations provide increased concentrations of Compound I for absorption; hence higher bioavailability.

The pharmaceutical industry is faced with the challenge of developing formulations for an increasing number of active molecules that possess low aqueous solubility and/or intestinal epithelial permeability. In some cases, as in the case of Compound I, acceptable bioavailability can not be readily achieved by means of traditional tablet or capsule formulations. An alternative dosage form for compounds with high lipid solubility is a lipid-based liquid-filled capsule (LFC). Such formulations have exhibited enhanced oral bioavailability and increased the interest in the potential of lipid-based formulations for oral administration. The exact mechanisms responsible for the enhanced bioavailability of poorly water soluble compounds are difficult to elucidate, but lipid-based formulations primarily increase exposure by overcoming the slow dissolution step from a solid dosage form (Pouton, C. W., Europ. J. Pharm. Sciences, 11 Suppl. 2 (2000) S93-S98). Additionally, these formulations may also enhance permeability (Aungst, B. J., J. Pharm. Sciences, 89:4 (2000) 429-442).

These lipid/surfactant vehicles form emulsions (i.e., suspensions of an oil droplet phase in an aqueous continuum phase) or microemulsions (i.e., a stable microstructured continuous phase) in aqueous environments. These are referred to in the literature as self-emulsifying drug delivery systems (SEDDS), S. Charman, et al., Pharm Res., vol. 9, 87 (1992). They are typically mixtures of oil, typically medium or long chain triglycerides, and non-ionic emulsifier that produced emulsions when dispersed in aqueous media, such as in the stomach and intestine (C. W. Pouton, Adv. Drug Deliv. Rev, vol. 25, 47 (1997); P. P. Constantinides, Pharm. Res., vol. 12, 1561 (1995); A. Humberstone and W. Charman, Adv. Drug Del. Rev., vol 25, 103 (1997)).

The formation of microemulsions has been linked to enhanced bioavailability of such formulations. In the case of cyclosporin A, the drug was more available from the NEORAL microemulsion formulation than the coarsely emulsifying SANDIMMNE formulation (Mueller, E. A., Kovarik, J. M., Van Bree, J. B., Tetzloff, W., Kutz, K., Pharm. Res., 11 (1994) 301-304). Self-emulsifying systems depend on the initial emulsification process to produce a dispersion. Self emulsifying formulations of cholesteryl ester transfer inhibitors are described in WO 03/000295.

Therefore, there remains a need to develop oral formulations of Compound I that would maximize exposure and reduce potential variability in absorption due to the food effect. A formulation that permits the presentation of larger doses per capsule would also be a desirable result. This invention provides pharmaceutical compositions that are liquid solutions, semisolids, suspensions, and (oil-in-water) emulsions of Compound I, said solutions being orally administrable. The solutions or dispersions may be administered, for example, as fill in encapsulated dosage forms such as liquid filled and sealed hard gelatin capsules or soft gelatin capsules containing plasticizers, such as glycerin and sorbitol. Compound I can be dissolved or dispersed in a variety of lipophilic vehicles, as further described and discussed below, such as digestible oils, cosolvents and surfactants, including mixtures of any two or more of the aforementioned vehicles.

### SUMMARY OF THE INVENTION

The present invention relates to pharmaceutical compositions for the oral administration of N-[1S,2S]-3-(4-chlorophenyl)-2-(3-cyanophenyl)-1-methylpropyl]-2-methyl-2-{[5-trifluoromethyl]pyridine-2-yl}oxy}propanamide (Compound I), a compound with low aqueous solubility (<0.4 μg/mL). When dosed as a crystalline solid, this compound was found to be very poorly orally bioavailable in dogs and monkeys, even when surfactant was included in the formulation to increase in vivo compound solubility. It has been found that oral bioavailability is surprisingly increased dramatically by using a liquid-filled capsule dosage form in which the compound is in solution in various combinations of liquid and semi-solid carriers, which include (a) digestible oils, including medium chain triglycerides, such as MIGLYOL 812, or 810 (triglycerides of caprylic/capric fatty acids, from SASOL), CAPTEX 355 (from Abitec Corp.), and CRODAMOL GTCC-PN (from Croda), and natural oils such as olive oil, corn oil, soybean oil, sesame oil, peanut oil, cottonseed oil and safflower oil; (b) lipophilic, low HLB surfactants, which include medium chain mono- and di-glycerides, such as IMWITOR 742 (mono- and di-glycerides of caprylic/capric fatty acids, from SASOL), and CAPMUL (from Abitec Corp.), as well as glycolized glycerides, such as LABRAFIL M 1944 CS (oleoyl macrogol glycerides by Gattefosse), and LABRAFIL M 2125 CS (linoleoyl macrogol glycerides by Gattefosse), and sorbitan fatty acid esters such as SPAN 80 (sorbitan monooleate from Uniqema, ICI group); (c) hydrophilic, high HLB surfactants, which include: Polysorbate 80-polyoxyethylene (20) sorbitan monooleate (also called TWEEN 80), and, in particular, CRILLET 4 HP (from Croda), polyoxyl 40 hydrogenated castor oil (CREMOPHOR RH40 from BASF), polyoxyl 35 castor oil (CREMOPHOL EL from BASF), and LABRASOL (caprylocaproyl macrogol glycerides from Gattefosse); and (d) cosolvents such as propylene glycol (PG), glycerol, ethanol, oleic acid, and polythethylene glycols such as PEG 400. A particular composition of the present invention, used to fill hard or soft gelatin capsules comprises: 0.8% to 2.4% Compound I, 48.7% to 49.6% Polysorbate 80, 48.7% to 49.6% IMWITOR 742, and 0.06% butylated hydroxyanisole.

Compound I is an inverse agonist of the Cannabinoid-1 (CB1) receptor, and compositions of the present invention comprising Compound I are useful in the treatment, prevention, and suppression of diseases mediated by the Cannabinoid-1 (CB1) receptor, including psychosis; memory deficits; cognitive disorders; Inigraine; neuropathy; neuro-inflammatory disorders including multiple sclerosis and Guillain-Barre syndrome and the inflammatory sequelae of viral encephalitis, cerebral vascular accidents, and head trauma; anxiety disorders; stress; epilepsy; Parkinson's disease; movement disorders; schizophrenia; substance abuse disorders, particularly of opiates, alcohol, marijuana, and nicotine, including smoking cessation; obesity; eating disorders associated with excessive food intake and complications associated therewith; constipation; chronic intestinal pseudo-obstruction; cirrhosis of the liver; and asthma.

### BRIEF DESCRIPTION OF THE DRAWINGS

FIG. 1 is a graph of the mean plasma concentration of Compound I over time generated in the experiment described in EXAMPLE 3, wherein male Rhesus monkeys were orally administered a 50 mg dosage of Compound I in a liquid filled gelatin capsule in several pharmaceutical carriers: 70:30 wt. % IMWITOR 742: TWEEN 80; 15:65:20 wt. % IMWITOR 742: MIGLYOL 812: TWEEN 80; 50:50 wt. % IMWITOR 742: TWEEN 80; 30:50:20 wt. % IMWITOR 742: MIGLYOL 812: TWEEN 80; and MIGLYOL 812. The data from this graph are also captured in the table in EXAMPLE 2, in Table 2.

FIG. 2 is a graph of the mean plasma concentration of Compound I over time generated in the experiment described in EXAMPLE 3 by administration of 50 mg Compound I in gelatin capsules to male Rhesus Monkeys in two formulations: Lactose/TWEEN 80 capsule and a MIGLYOL 812 capsule. The data from this graph are also captured in the table in EXAMPLE 2, in Table 2.

### DETAILED DESCRIPTION OF THE INVENTION

The present invention relates to pharmaceutical compositions for the oral administration of N-[1S,2S]-3-(4-chlorophenyl)-2-(3-cyanophenyl)-1-methylpropyl]-2-methyl-2-{[5-trifluoromethyl]pyridine-2-yl}oxy}propanamide, (Compound I) a compound with low aqueous solubility (<0.4 μg/mL). When dosed as a crystalline solid, this compound was found to be very poorly orally bioavailable in dogs and monkeys, even when surfactant was included in the formulation to increase in vivo compound solubility. It has been surprisingly found that oral bioavailability is increased dramatically by using a liquid-filled capsule dosage form in which the Compound I or a pharmaceutically acceptable salt or solvate thereof is in solution in various combinations of liquid and semi-solid carriers which include (a) digestible oils; (b) lipophilic, low HLB surfactants; (c) hydrophilic, high HLB surfactants; and (d) cosolvents.

One embodiment of the present invention comprises: Compound I or a pharmaceutically acceptable salt or solvate thereof and the combination of a low HLB surfactant and a high HLB surfactant. Another embodiment of the present invention comprises: Compound I and the combination of a pharmaceutically acceptable digestible oil and a cosolvent which is miscible therewith. In yet another embodiment of the present invention is Compound I and the combination of a low HLB surfactant, a high HLB surfactant, and a digestible oil. In still another embodiment of the present invention comprises Compound I and a combination of a low HLB surfactant, a high HLB surfactant and a preservative.

One embodiment of the present invention is a composition comprising Compound I and a lipophilic vehicle selected from digestible oils, lipophilic solvents (also referred to herein as a “cosolvents”, whether or not another solvent is in fact present), solvents, surfactants and mixtures of any two or more thereof.

In another embodiment of the present invention is a composition comprising: Compound I, and a pharmaceutically acceptable carrier selected from: a high HLB surfactant, a low HLB surfactant, a digestible oil, and a cosolvent.

In another embodiment of the present invention is a composition comprising: Compound I, and a pharmaceutically acceptable carrier selected from: a high HLB surfactant, a low HLB surfactant, a digestible oil, and a cosolvent, and mixtures of any two or more thereof.

In one class of this embodiment, N-[1S,2S]-3-(4-chlorophenyl)-2-(3-cyanophenyl)-1-methylpropyl]-2-methyl-2-{[5-trifluoromethyl]pyridine-2-yl}oxy}propanamide, is unsolvated. In another class of this embodiment, the N-[1S,2S]-3-(4-chlorophenyl)-2-(3-cyanophenyl)-1-methylpropyl]-2-methyl-2-{[5-trifluoromethyl]pyridine-2-yl}oxy}propanamide, is a solvate or a hemisolvate.

In another class of this embodiment, the N-[1S,2S]-3-(4-chlorophenyl)-2-(3-cyanophenyl)-1-methylpropyl]-2-methyl-2-{[5-trifluoromethyl]pyridine-2-yl}oxy}propanamide, is the unsolvated free base.

In yet another class of this embodiment, the N-[1S,2S]-3-(4-chlorophenyl)-2-(3-cyanophenyl)-1-methylpropyl]-2-methyl-2-{[5-trifluoromethyl]pyridine-2-yl}oxy}propanamide, is the unsolvated salt.

In still another class of this embodiment, the N-[1S,2S]-3-(4-chlorophenyl)-2-(3-cyanophenyl)-1-methylpropyl]-2-methyl-2-{[5-trifluoromethyl]pyridine-2-yl}oxy}propanamide, is the unsolvated HCl salt.

In another class of this embodiment, the N-[1S,2S]-3-(4-chlorophenyl)-2-(3-cyanophenyl)-1-methylpropyl]-2-methyl-2-{[5-trifluoromethyl]pyridine-2-yl}oxy}propanamide, is the solvated salt.

In yet another class of this embodiment, the N-[1S,2S]-3-(4-chlorophenyl)-2-(3-cyanophenyl)-1-methylpropyl]-2-methyl-2-{[5-trifluoromethyl]pyridine-2-yl}oxy}propanamide, is the solvated HCl salt.

In one class of this embodiment, the high HLB surfactant is selected from hydrophilic surfactants having an HLB of 8-20. In one subclass of this class, the high HLB surfactant has an HLB greater than 10. In another subclass of this class, the high HLB surfactant is selected from: nonionic surfactants, such as polyoxyethylene 20 sorbitan monooleate, Polysorbate 80, sold under the trademark TWEEN 80, available commercially from ICI and CRILLET 4 NF and CRILLET 4 HP from Croda; polyoxyethylene 20 sorbitan monolaurate (Polysorbate 20, TWEEN 20) available as CRILLET 1 NF and CRILLET 1 HP from Croda; polyethylene (40 or 60) hydrogenated castor oil (available under the registered trademarks CREMOPHOR RH40 and RH60 from BASF); polyoxyethylene (35) castor oil (CREMOPHOR EL from BASF and ETOCAS 30 from Croda)); polyethylene (60) hydrogenated castor oil (NIKKOL HCO-60); alpha tocopheryl polyethylene glycol 1000 succinate (Vitamin E TPGS from Eastman); glyceryl PEG 8 caprylate/caprate (caprylocaproyl macrogol glycerides available commercially under the registered trademark LABRASOL from Gattefosse, and under the tradename ACCONON MC-8 from Abitec Corp.); PEG 32 glyceryl laurate (lauroyl macrogol glycerides sold commercially under the registered trademark GELUCIRE 44/14 from Gattefosse); stearyl macrogol glycerides commercially under the registered trademark GELUCIRE 50/13 from Gattefosse; polyoxyethylene fatty acid esters (available commercially under the registered trademark MYRJ from ICI); polyoxyethylene fatty acid ethers (available commercially under the registered trademark BRIJ from ICI); and Poloxamers (124, 188, 407) available by the trade names of LUTROLS or PLURONICS from BASF. In one subclass, the high HLB surfactant is selected from: Polysorbate 80, including CRILLET 4 NF, CRILLET 4 HP, and TWEEN 80; CREMOPHOR RH40; LABRASOL; and Vitamin E TPGS. In yet another subclass, the high HLB surfactant is Polysorbate 80, particularly CRILLET 4 BP.

In another class of this embodiment, the low HLB surfactant is selected from: lipophilic surfactants having an HLB of less than 8. In one subclass of this class, the lipophilic surfactant is selected from: mono and diglycerides; more specifically mono- and di-glycerides of capric and caprylic acids available under the following registered trademarks: CAPMUL MCM, MCM 8, and MCM 10, available commercially from Abitec, and IMWITOR 988, 928, 780K, 742, 380, or 308, available commercially from SASOL; oleoyl macrogol glycerides, available under the registered trademark LABRAFIL M 1944 CS from Gattefosse; (oleoyl macrogol glycerides) polyoxyethylene corn oil, available commercially as LABRAFIL M 2125 from Gattefosse; propylene glycol monocaprylate commercially under the registered trademark CAPRYOL 90 (from Gattefosse); PEG-caprylic/capric glycerides (SOFTIGENs, available from SASOL); propylene glycol monolaurate, available commercially as LAUROGLYCOL from Gattefosse; propylene glycol dicaprylate/caprate available commercially as CAPTEX 200 from Abitec; or MIGLYOL 840 from SASOL. Other low HLB materials include polyglyceryl oleate available commercially as PLUROL OLEIQUE CC497 oleique from Gattefosse; glyceryl oleate available as PECEOL from Gattefosse; glyceryl linoleate available as MAISINE 35-1 from Gattefosse; sorbitan esters of fatty acids (e.g., sorbitan monooleate available under the registered trademark SPAN 80 from Uniqema/ICI and CRILL 4 NF from Croda; sorbitan laurate available under the registered trademark SPAN 20 from Uniqema/ICI; and CRILL 1 NF from Croda). Preferred from this class are IMWITOR 742, PECEOL, CAPMUL MCM, SPAN 80, and LABRAFIL M1944 CS. Most preferred is IMWITOR 742 by SASOL.

In still another class of this embodiment, the digestible oil or fat (liquid or semi-solid vehicle is selected from: medium chain triglycerides (MCT, C6-C12), long chain triglycerides (LCT, C14-C20), and mixtures of mono-, di- and tri-glycerides, or lipophilic derivatives of fatty acids. Examples of MCT's useful in the present invention include fractionated coconut oils, such as MIGLYOL 812 which is a 56% caprylic (C8) and 36% capric (C10) triglyceride, MIGLYOL 810 (68% C8 and 28% C10), NEOBEE MS, CAPTEX 300, CAPTEX 355, LABRAFAC CRODAMOL GTCC, SOFTISANS 100, SOFTISANS 142, SOFTISANS 378, and SOFTISANS 649. The MIGLYOLs are supplied by SASOL, NEOBEE by Stepan Europe, CAPTEX by Abitec Corp., LABRAFAC by Gattefosse, and CRODAMOL by Croda Corp. Examples of LCTs useful in the compositions of the present invention include vegetable oils such as soybean, safflower, corn, olive, cottonseed, arachis, sunflowerseed, palm, and rapeseed. Examples of fatty acid esters of alkyl alcohols useful in the present invention include ethyl oleate and glyceryl monooleate. In one aspect of the present invention, the digestible oil is selected from olive oil, corn oil, soybean oil, and MIGLYOL 812. In one subclass of this class, the digestible oil is a MCT. In another subclass, the digestible oil is MIGLYOL 812. Examples of semisolid vehicles include glyceryl monostearate (commercially available as IMWITOR 491 from SASOL), glycerol esters of fatty acids (such as GELUCIRE 33/01, GELUCIRE 39/01, and GELUCIRE 43/01, available from Gattefosse) and fatty acid esters such as SOFTISANS(SOFTISAN 100, SOFTISAN 142, SOFTISAN 378, and SOFTISAN 649 available from SASOL).

In yet another class, the cosolvent is selected from: triacetin (1,2,3-propanetriyl triacetate or glyceryl triacetate available from Eastman Chemical Corp.) or other polyol esters of fatty acids, trialkyl citrate esters, propylene carbonate, dimethylisosorbide, ethyl lactate, N-methylpyrrolidones, diethylene glycol monoethyl ether (TRANSCUTOL by Gattefosse), peppermint oil, 1,2-propylene glycol (PG), ethanol, oleic acid, and polyethylene glycols. In one subclass of this class, the cosolvent is selected from: triacetin, propylene carbonate (Huntsman Corp.), transcutol (Gattefosse), ethyl lactate (Purac, Lincolnshire, Nebr.), propylene glycol, oleic acid, dimethylisosorbide (sold under the registered trademark ARLASOLVE DMI, ICI Americas), steryl alcohol, cetyl alcohol, cetosteryl alcohol, glyceryl behenate, and glyceryl palmitostearate. In another subclass, a cosolvent selected from propylene glycol, ethanol, and oleic acid is employed.

Reference to a compositional component such as a “digestible oil”, to a “surfactant” and so forth, shall be understood as including mixtures of such components such as mixtures of digestible oils and surfactants.

Reference to a specific weight or percentage of “active ingredient”, Compound I, or N-[1S,2S]-3-(4-chlorophenyl)-2-(3-cyanophenyl)-1-methylpropyl]-2-methyl-2-{[5-trifluoromethyl]pyridine-2-yl}oxy}propanamide, is on the basis of the free base weight, absent the weight of any counterion or solvate present, unless otherwise indicated. For example, the phrase “1 mg N-[1S,2S]-3-(4-chlorophenyl)-2-(3-cyanophenyl)-1-methylpropyl]-2-methyl-2-{[5-trifluoromethyl]pyridine-2-yl}oxy}propanamide MTBE hemisolvate” means that the amount of the compound selected is based on 1 mg of N-[1S,2S]-3-(4-chlorophenyl)-2-(3-cyanophenyl)-1-methylpropyl]-2-methyl-2-{[5-trifluoromethyl]pyridine-2-yl}oxy}propanamide as the free base, absent the weight of the solvent present in the solvate.

In one embodiment of the present invention, Compound I is dissolved or dispersed in a high HLB surfactant. In another embodiment, Compound I is dissolved or dispersed in a high HLB surfactant or surfactant mixture which surfactant mixture may optionally contain one or more low HLB surfactants. In still another embodiment, Compound I is dissolved or dispersed in a digestible oil, such as a medium chain triglyceride or a mixture of digestible oils. In yet another embodiment, Compound I is dissolved or dispersed in a pharmaceutically acceptable lipophilic solvent optionally containing a digestible oil or digestible oil mixture. In another embodiment of the present invention, Compound I is dissolved or dispersed in a low HLB surfactant or surfactant mixture which surfactant mixture may optionally contain one or more high HLB surfactants. In still another embodiment, Compound I is dissolved or dispersed in a pharmaceutically acceptable mixture of a high HLB surfactant and a low HLB surfactant. In a class of this embodiment the high HLB surfactant and low HLB surfactant are present in equal amounts by weight in the mixture. In still another embodiment, Compound I is dissolved or dispersed in a cosolvent.

The presence of one or more surfactants can, upon contacting the pharmaceutical composition with water, yield an emulsion that is either preformed by mixing with an aqueous phase or that is generated in vivo by contacting the aqueous fluids of the gastrointestinal tract. Formation of an emulsion can improve bioavailability and may reduce the food effect in man (i.e., the effect of food upon absorption and/or bioavailability of a drug). It can also allow the oil to be consumed as a beverage in addition to being administered in capsules. Use of surfactants to provide an emulsion can also be of value for increasing exposures in toxicology species. Addition of a cosolvent to a pharmaceutical composition comprising Compound I and a pharmaceutically acceptable carrier selected from a high HLB surfactant, a low HLB surfactant, a digestible oil, and a cosolvent can have the advantage of higher solubility and thus a higher dose in a given volume of formulation than is obtainable without the cosolvent. It is advantageous for bioavailability to have the entire dose dissolved. The presence of a third component in any of the above embodiments may also improve miscibility between the first two components.

In a particularly preferred embodiment, the invention provides a composition of matter for increasing the oral bioavailability of Compound I. The composition comprises: 1. Compound I; 2. a surfactant having an HLB of from 1 to not more than 8; 3. a surfactant having an HLB of over 8 up to 20; and 4. optionally, a digestible oil. Optionally, an antioxidant may also be present. In such formulations, all of the excipients are pharmaceutically acceptable. The above composition is sometimes referred to herein as a “pre-concentrate”, in reference to its function of forming a stable emulsion when gently mixed with water or other aqueous medium, usually gastrointestinal fluids. It is also referred to herein as a “fill”, referring to its utility as a fill for a hard gelatin or soft gelatin capsule.

Reference herein is frequently made to a soft gelatin capsule as a preferred dosage form for use with this invention, “softgel” being an abbreviation for soft gelatin capsules. It is understood that when reference is made to the term “softgel” alone, it shall be understood that the invention applies equally to all types of gelatin and non-gelatin capsules, regardless of hardness, softness, and so forth. In one embodiment of the present invention, the soft gelatin capsule contains plasticizers, such as glycerin and sorbitol. Colorant may be added to the gel mixture prior to encapsulation to produce soft gelatin capsules of the desired hue.

As noted above, and as discussed further below, a digestible oil can form a part of the pre-concentrate. If no other component of the pre-concentrate is capable of functioning as an emulsifiable oily phase, a digestible oil can be included as the oil which acts as a solvent for Compound I and which disperses to form the (emulsifiable) oil droplet phase once the pre-concentrate has been added to water. Some surfactants can serve a dual function, however, i.e., that of acting as a surfactant and also as a solvent and an oily vehicle for forming an oil-in-water emulsion. In the event such a surfactant is employed, and, depending on the amount used, a digestible oil may be required in less of an amount, or not required at all.

The pre-concentrate can be self-emulsifying or self-microemulsifying. The term “self-emulsifying” refers to a formulation which, when diluted by a factor of at least 100 by water or other aqueous medium and gently mixed, yields an opaque, stable oil/water emulsion with a mean droplet diameter less than about 5 microns, but greater than 100 nm, and which is generally polydisperse. The term “self-microemulsifying” refers to a pre-concentrate which, upon at least 100× dilution with an aqueous medium and gentle mixing, yields a non-opaque, stable oil/water emulsion with an average droplet size of about 1 micron or less, said average particle size preferably being less than 100 nm. The particle size is primarily unimodal. Both self-emulsifying and self-microemulsifying formulations are encompassed by the present invention.

“Gentle mixing” as used above is understood in the art to refer to the formation of an emulsion by gentle hand (or machine) mixing, such as by repeated inversions on a standard laboratory mixing machine. High shear mixing is not required to form the emulsion. Such pre-concentrates generally emulsify nearly spontaneously when introduced into the human (or other animal) gastrointestinal tract.

Combinations of two surfactants, one being a low HLB surfactant with an HLB of 1 to 8, the other being a high HLB surfactant with a higher HLB of over 8 to 20, preferably 9 to 20, can be employed to create the right conditions for efficient emulsification. The HLB, an acronym for “hydrophobic-lipophilic balance”, is a rating scale which can range from 1-20 for non-ionic surfactants. The higher the HLB; the more hydrophilic the surfactant. Hydrophilic surfactants (HLB 8-20), when used alone, provide fine emulsions which are, advantageously, more likely to empty uniformly from the stomach and provide a much higher surface area for absorption. Disadvantageously, however, limited miscibility of such high HLB surfactants with oils can limit their effectiveness, and thus a low HLB, lipophilic surfactant (HLB 1-8) may also be included in the compositions of the present invention. This combination of surfactants can also provide superior emulsification.

Hydrophilic surfactants having an HLB of 8-20, preferably having an HLB greater than 10, can be used alone as the vehicle or in a vehicle which includes a hydrophilic surfactant as part of a mixture, and are particularly effective at reducing emulsion droplet particle size. Suitable choices include nonionic surfactants such as polyoxyethylene 20 sorbitan monooleate; polysorbate 80, sold under the trademark TWEEN 80, available commercially from ICI, and CRILLET 4 NF and CRILLET 4 HP from Croda; polyoxyethylene 20 sorbitan monolaurate (Polysorbate 20, TWEEN 20) available as CRILLET 1 NF and CRILLET 1 HP from Croda; polyethylene (40 or 60) hydrogenated castor oil (available under the registered trademarks CREMOPHOR RH40 and RH60 from BASF); polyoxyethylene (35) castor oil (CREMOPHOR EL from BASF and ETOCAS 30 from Croda); polyethylene (60) hydrogenated castor oil (IKOL HCO-60); alpha tocopheryl polyethylene glycol 1000 succinate (Vitamin E TPGS); glyceryl PEG 8 caprylate/caprate (caprylocaproyl macrogol glycerides available commercially under the registered trademark LABRASOL from Gattefosse, and under the tradename ACCONON MC-8 from Abitec Corp.); PEG 32 glyceryl laurate (lauroyl macrogol glycerides sold commercially under the registered trademark GELUCIRE 44/14 from Gattefosse); stearyl macrogol glycerides commercially under the registered trademark GELUCIRE 50/13 from Gattefosse; polyoxyethylene fatty acid esters (available commercially under the registered trademark MYRJ from ICI); polyoxyethylene fatty acid ethers (available commercially under the registered trademark BRIJ from ICI); Poloxamers (124, 188, 407) available by the trade names of Lutrols or Pluronics from BASF, and Imwitor 380, 780K, 928 (from SASOL). In one subclass the high HLB surfactant is selected from: TWEEN 80, CREMOPHOR RH40, LABRASOL and Vitamin E TPGS. In yet another subclass, the high HLB surfactant is Polysorbate 80 and, in particular, CRILLET 4 HP.

Lipophilic surfactants having an HLB of less than 8 can be used alone as the vehicle, or in a vehicle which includes a lipophilic surfactant as part of a mixture, and are useful for achieving a balance of polarity to provide a stable emulsion, and have also been used to reverse the lipolysis inhibitory effect of hydrophilic surfactants. Suitable lipophilic surfactants include mono- and diglycerides; more specifically mono- and di-glycerides of capric and caprylic acid available under the following registered trademarks: CAPMUL MCM, MCM 8, and MCM 10, available commercially from Abitec; and IMWITOR 988, 928, 780K, 742, 380, or 308, available commercially from SASOL; oleoyl macrogol glycerides, available under the registered trademark LABRAFIL M 1944 CS from Gattefosse; polyoxyethylene corn oil, available commercially as LABRAFIL M 2125 from Gattefosse; propylene glycol caprylate available commercially under the registered trademark CAPRYOL PGMC (from Gattefosse); propylene glycol monocaprylate commercially under the registered trademark CAPRYOL 90 (from Gattefosse); PEG-caprylic/capric glycerides (SOFTIGENs, available by SASOL); propylene glycol monolaurate, available commercially as LAUROGLYCOL from Gattefosse; and propylene glycol dicaprylate/caprate available commercially as CAPTEX 200 from Abitec or MIGLYOL 840 from SASOL. Other low HLB materials include: polyglyceryl oleate available commercially as PLUROL OLEIQUE CC 497 oleique from Gattefosse; sorbitan glyceryl oleate available as MAISINE 35-1 from Gattefosse; and sorbitan esters of fatty acids (e.g. sorbitan monooleate available under the registered trademark SPAN 80 from Uniqema/ICI and CRILL 4 NF from Croda, sorbitan laurate available under the registered trademark SPAN 20 from Uniqema/ICI and CRILL 1 NF from Croda). Preferred from this class are IMWITOR 742, PECEOL, CAPMUL MCM, SPAN 80 and LABRAFIL M1944 CS. Most preferred is IMWITOR 742 by SASOL.

Suitable digestible oils or fats (liquid or semi-solid vehicles), which can be used alone as the vehicle or in a vehicle which includes a digestible oil as part of a mixture, include medium chain triglycerides (MCT, C6-C12) and long chain triglycerides (LCT, C14-C20), and mixtures of mono-, di-, and triglycerides, or lipophilic derivatives of fatty acids such as esters with alkyl alcohols. Examples of preferred MCT's include fractionated coconut oils, such as MIGLYOL 812 which is a 56% caprylic (C8) and 36% capric (C10) triglyceride, MIGLYOL 810 (68% C8 and 28% C10), NEOBEE MS, CAPTEX 300, CAPTEX 355, LABRAFAC CRODAMOL GTCC. The MIGLYOLs are supplied by SASOL NEOBEE by Stepan Europe, CAPTEX by Abitec Corp., LABRAFAC by Gattefosse, and CRODAMOL by Croda Corp. Examples of LCTs include vegetable oils such as soybean, safflower, corn, olive, cottonseed, arachis, sunflowerseed, palm, and rapeseed. Examples of fatty acid esters of alkyl alcohols include ethyl oleate and glyceryl monooleate. In one aspect of the present invention, the digestible oil is selected from olive oil, corn oil, soybean oil, and MIGLYOL 812. Of the digestible oils MCT's are preferred, and MIGLYOL 812 is most preferred. Examples of semisolid vehicles include glyceryl monostearate (commercially available as IMWITOR 491 by SASOL), glycerol esters of fatty acids (such as GELUCIRE 33/01, GELUCIRE 39/01 and GELUCIRE 43/01, available from Gattefosse), and fatty acids esters such as SOFTISANS(SOFTISAN 100, SOFTISAN 142, SOFTISAN 378, and SOFTISAN 649 available from SASOL).

The vehicle may also be a pharmaceutically acceptable solvent, for use alone, or as a cosolvent in a mixture. Suitable solvents/cosolvents include any solvent that is used to increase solubility of Compound I in the formulation in order to allow delivery of the desired dose per dosing unit or to enhance the miscibility of the various formulation components. Suitable solvents include triacetin (1,2,3-propanetriyl triacetate or glyceryl triacetate available from Eastman Chemical Corp.) or other polyol esters of fatty acids, trialkyl citrate esters, propylene carbonate, dimethylisosorbide, ethyl lactate, N-methylpyrrolidones, diethylene glycol monoethyl ether (TRANSCUTOL by Gattefosse), peppermint oil, 1,2-propylene glycol (PG), ethanol, oleic acid, and polyethylene glycols. Preferred as solvents are triacetin, propylene carbonate (Huntsman Corp.), TRANSCUTOL (Gattefosse), ethyl lactate (Purac, Lincolnshire, Nebr.), propylene glycol, oleic acid, dimethylisosorbide (sold under the trademark ARLASOLVE DMI, ICI Americas) steryl alcohol, cetyl alcohol, cetosteryl alcohol, glyceryl behenate, and glyceryl palmitostearate. In one embodiment, a cosolvent selected from propylene glycol, and oleic acid is employed. A hydrophilic solvent is more likely to migrate to the capsule shell and soften the shell, and, if volatile, its concentration in the composition can be reduced, but with a potential negative impact on active component solubility. In one embodiment of the present invention, the cosolvent is selected from oleic acid, propylene glycol and ethanol.

In one embodiment of the present invention is a composition comprising, Compound I, and a carrier selected from a high HLB surfactant selected from TWEEN 80, CRILLET 4 HP, and CREMOPHOR EL; a low HLB surfactant selected from: IMWITOR 742, PECEOL, CAPMUL MCM, SPAN 80 and LABRAFIL M1944 CS; a digestible oil is selected from: olive oil, corn oil, soybean oil, and MIGLYOL 812; and a cosolvent is selected from: propylene glycol, ethanol, and oleic acid.

The composition can be formulated as a fill encapsulated in a gelatin capsule of appropriate gelatin composition, a hard gelatin capsule with an appropriate seal, a non-gelatin capsule such as a hydroxypropyl methylcellulose capsule, or an oral liquid or emulsion by methods commonly employed in the art. In one embodiment of the present invention, the fill is encapsulated in a sealed hard gelatin capsule or a soft gelatin capsule containing plasticizers, such as glycerin and sorbitol. In one class of this embodiment, the hard gelatin capsule is sealed by band sealing using a gelatin ribbon, or LEMS (i.e., spraying with a hydroalcoholic solution to locally melt and seal the gelatin capsule pieces). The fill is prepared by mixing the excipients and Compound I with heating if required.

Another embodiment of the present invention comprises a capsule comprising Compound I, and a pharmaceutically acceptable carrier selected from:
(a) a high HLB surfactant selected from CREMOPHOR EL and Polysorbate 80, selected from TWEEN 80, CRILLET 4 HP, CRILLET 4 NF;
(b) a low HLB surfactant selected from: IMWITOR 742, PECEOL, CAPMUL MCM, CAPRYOL, SPAN 80 and LABRAFIL M1944 CS;
(c) a digestible oil selected from: olive oil, corn oil, soybean oil, and MIGLYOL 812; and
(d) a cosolvent selected from: propylene glycol, ethanol, and oleic acid.

The ratio of Compound I, surfactants, digestible oils, and/or cosolvents depends upon the efficiency of emulsification and the solubility, and the solubility depends on the dose per capsule that is desired. In general, the following ranges, in weight percent, of the components for a formulation of Compound I are: 0.01-50% Compound I; 0-99.99% cosolvent; 0-99.99% high HLB surfactant; 0-99.99% low HLB surfactant and 0-99.99% digestible oil. In one class having advantageous bioavailability are those wherein the ratio of components are: 0.01-25% Compound I; 0-70% digestible oil; 0-50% high HLB surfactant; 0-70% low HLB surfactant. In one subclass of this class are formulations with ranges of 0.01-13% Compound I; 20-50% high HLB surfactant; 40-80% low HLB surfactant. In another subclass of this class are formulations with ranges of 0.8% to 2.4% Compound I, 48.7% to 49.6% high HLB surfactant, 48.7% to 49.6% low HLB surfactant, and optionally 0.06% antioxidant.

In particular, a composition of the present invention comprises Compound I dissolved in a 1:1 mixture by weight of Polysorbate 80 and IMWITOR 742; particularly TWEEN 80 and IMWITOR 742, or CRILLET 4 HP and IMWITOR 742. This composition has the advantage of increased bioavailability, increased potency, good safety and tolerability, and ease of processing.

In addition to the main softgel capsule ingredients previously noted, other stabilizing additives, as conventionally known in the art of softgel formulation, can be introduced to the fill as needed, usually in relatively small quantities, such as antioxidants (BHA (butylated hydroxyanisole), BHT (t-butylhydroxytoluene), tocopherol, propyl gallate, etc.) and other preservatives such as benzyl alcohol or parabens. Preferably, the antioxidant or preservative is present in a weight percent range of 0.01% to 0.1%.

A particular composition of the present invention, used to fill hard or soft gelatin capsules comprises: 0.8% to 2.4% Compound I, 48.7% to 49.6% Polysorbate 80, 48.7% to 49.6% IMWITOR 742, and 0.06% butylated hydroxyanisole.

The composition can be formulated as a fill encapsulated in a soft gelatin capsule, a hard gelatin capsule with an appropriate seal, a non-gelatin capsule such as a hydroxypropyl methylcellulose capsule or an oral liquid or emulsion by methods commonly employed in the art. The fill is prepared by mixing the excipients and Compound I optionally with heating if required.

The present invention is also related to a process for preparing a capsule, comprising the steps of:
(a) blending the carrier ingredients selected from a high HLB surfactant, a low HLB surfactant and a digestible oil to form a vehicle;
(b) dissolving the N-[1S,2S]-3-(4-chlorophenyl)-2-(3-cyanophenyl)-1-methylpropyl]-2-methyl-2-{[5-trifluoromethyl]pyridine-2-yl}oxy}propanamide or a pharmaceutically acceptable salt or solvate thereof in the vehicle to form a solution or dispersion; and
(c) encapsulating the solution or dispersion of N-[1S,2s]-3-(4-chlorophenyl)-2-(3-cyanophenyl)-1-methylpropyl]-2-methyl-2-{[5-trifluoromethyl]pyridine-2-yl}oxy}propanamide or the pharmaceutically acceptable salt or solvate thereof in the vehicle in appropriate hard or soft gelatin capsules.

Another aspect of the present invention relates to a process for preparing fill for a capsule comprising the steps of:
(a) heating a low HLB surfactant;
(b) adding Compound I to the heated low HLB surfactant and maintaining the heated temperature until the Compound I is dissolved;
(c) adding a high HLB surfactant to the solution of Compound I, and stirring.

In one embodiment of the present process, an antioxidant is added to the low HLB surfactant before heating. In another embodiment of the present invention, the low HLB surfactant is heated to 40+/−5° C. In another embodiment of the present process, the low HLB surfactant is IMWITOR 742. In anther embodiment, the antioxidant is BHA. In another embodiment of the present process, the high HLB surfactant is Polysorbate 80. In still another embodiment of the present invention, the solution comprising Compound I, low HLB surfactant, and high HLB surfactant, is filtered. In a class of this embodiment, the filtered solution is deaerated under vacuum.

The present invention is also related to a product produced by the process described above

The present invention is also related to a process for preparing a capsule, comprising the steps of:
(a) heating the low HLB surfactant;
(b) adding Compound I to the heated low HLB surfactant and maintaining the heated temperature until the Compound I is dissolved;
(c) adding the high HLB surfactant to the solution of Compound I, and stirring the resulting mixture; and
(d) encapsulating the mixture of Compound I in appropriate hard or soft gelatin capsules.

In one class of this embodiment, the solution is filtered through a 35 micron mesh filter.

In another class of this embodiment, the solution is deaerated under vacuum until visual examination reveals that all air is removed (at least one hour). In yet another class of this embodiment, the fill mixture encapsulated in a soft gelatin capsule.

Another embodiment of the process for preparing fill for a capsule of the present invention, comprises the steps of:
(a) adding BHA to IMWITOR 742 and heating to 40+/−5° C.;
(b) adding Compound I to the IMWITOR 742/BHA, and mixing at 40+/−5° C. until the Compound I is dissolved;
(c) adding Polysorbate 80 to the solution of Compound I, IMWITOR 742/BHA and mixing.

In one class of this embodiment, the solution is filtered through a 35 micron mesh filter. In another class of this embodiment, the solution is deaerated under vacuum until visual examination reveals that all air is removed (at least one hour). In yet another class of this embodiment, the fill mixture encapsulated in a soft gelatin capsule.

The present invention is also related to a process for preparing a capsule, comprising the steps of:
(a) adding BHA to a portion of the IMWITOR 742, and heating to 40+/−5° C.;
(b) adding Compound I to the IMWITOR 742/BHA, and mixing at 40+A 5° C. until the Compound I is dissolved;
(c) adding Polysorbate 80 and the remaining IMWITOR 742 to the solution of Compound I, IMWITOR 742/BHA and mixing; and
(d) encapsulating the mixture of Compound I in appropriate hard or soft gelatin capsules.

In one class of this embodiment, the solution is filtered through a 35 micron mesh filter. In another class of this embodiment, the solution is deaerated under vacuum until visual examination reveals that all air is removed (at least one hour). In yet another class of this embodiment, the fill mixture encapsulated in a soft gelatin capsule.

Oral delivery of Compound I is particularly difficult because its aqueous solubility is extremely low, typically being less than 0.4 ug/mL. Achieving therapeutic drug levels in the blood by oral dosing of practical quantities of a drug generally requires a large enhancement in drug concentrations in the gastrointestinal fluid and a resulting large enhancement in bioavailability. The formulations of this invention will be administered in such an amount that an effective dose of Compound I is administered to the patient. The amount of Compound I will generally be known or determined by the attending physician. Thus, the amount or volume of preconcentrate administered will be determined by the amount of Compound I prescribed and/or otherwise desired as a dose and the solubility of the Compound I in the preconcentrate. In general, an effective dose for Compound I is from 0.01 mg to about 1000 mg per day, in single or divided doses; preferably from about 0.1 mg to about 10 mg per day, in single or divided doses. For oral administration, the compositions are preferably provided in the form of liquid- or semi-solid-filled capsules containing from 0.01 to 1,000 mg, preferably 0.01, 0.05, 0.1, 0.5, 1, 2, 2.5, 3, 4, 5, 6, 7, 7.5, 8, 9, 10, 15, 20, 25, 30, 40, 50, 100, 250, 500, 750 or 1000, most preferably 2, 4, or 6 milligrams of the active ingredient for the symptomatic adjustment of the dosage to the patient to be treated.

The compositions of the invention are pre-concentrates for emulsification which are generally administered orally, in soft or hard gelatin capsules, gelatin encapsulation technology being well known to the pharmaceutical arts. Such pre-concentrates can also be administered in aqueous oral emulsions by adding the pre-concentrate to water or other aqueous liquid (e.g., soda). They can be mixed with an aqueous liquid and sold as pre-formed emulsions, or added to food such as ice cream.

Compositions of the present invention comprising Compound I are useful in the treatment, prevention and suppression of diseases mediated by the Cannabinoid-1 (CB1) receptor, including psychosis; memory deficits; cognitive disorders; migraine; neuropathy; neuro-inflammatory disorders including multiple sclerosis and Guillain-Barre syndrome and the inflammatory sequelae of viral encephalitis, cerebral vascular accidents, and head trauma; anxiety disorders; stress; epilepsy; Parkinson's disease; movement disorders; schizophrenia; substance abuse disorders, particularly to opiates, alcohol, marijuana, and nicotine, including smoking cessation; obesity; eating disorders associated with excessive food intake and complications associated therewith; constipation; chronic intestinal pseudo-obstruction; cirrhosis of the liver; and asthma.

In one embodiment of the present invention, the compositions are pharmaceutical compositions. In one class of this embodiment, the pharmaceutical composition is for use in treating obesity in a mammal.

In another class of this embodiment, the pharmaceutical composition is for use in treating obesity in a human.

In another embodiment of the present invention, the compositions are pharmaceutical compositions for use in treating substance abuse disorders. In one class of this embodiment, the substance abuse disorders are selected from abuse of opiates, alcohol, marijuana, and nicotine.

In another embodiment of the present invention, the pharmaceutical composition is provided for use in smoking cessation.

In yet another embodiment of the present invention, the pharmaceutical composition is provided for use in treating alcohol addiction.

In still another embodiment of the present invention, the pharmaceutical composition is for use in treating a patient with diabetes.

In yet another embodiment of the present invention, the pharmaceutical composition is for use in treating an obese human patient.

In still another embodiment of the present invention, the pharmaceutical composition is for use in treating a patient who smokes. In one class of this embodiment, the patient no longer wishes to continue smoking.

In another embodiment of the present invention, the pharmaceutical composition is for use in treating a patient who is abusing a substance selected from opiates, alcohol, and marijuana.

In yet another embodiment of the present invention, the pharmaceutical composition is for use in treating a patient who is an alcoholic.

The terms “administration of” and or “administering a” compound should be understood to mean providing the composition of the invention to the individual in need of treatment.

The administration of the composition of the present invention to practice the present methods of therapy is carried out by administering an effective amount of the compound of structural formula I to the patient in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk for obesity” is an otherwise healthy subject with a BMI of 25 kg/m² to less than 30 kg/m² or a subject with at least one co-morbidity with a BMI of 25 kg/m² to less than 27 kg/m².

Obesity-induced or obesity-related co-morbidities include, but are not limited to, diabetes, non-insulin dependent diabetes mellitus-type 2, impaired glucose tolerance, impaired fasting glucose, insulin resistance syndrome, dyslipidemia, hypertension, hyperuricacidemia, gout, coronary artery disease, myocardial infarction, angina pectoris sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions. The compositions of the present invention are useful for treating patients with obesity-induced or obesity-related co-morbidities, as defined above.

“Treatment of obesity and obesity-related disorders” refers to the administration of the compositions of the present invention to reduce or maintain the body weight of an obese subject. One outcome of treatment may be reducing the body weight of an obese subject relative to that subject's body weight immediately before the administration of the compositions of the present invention. Another outcome of treatment may be preventing regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention of obesity and obesity-related disorders” refers to the administration of the compositions of the present invention to reduce or maintain the body weight of a subject at risk for obesity. One outcome of prevention may be reducing the body weight of a subject at risk for obesity relative to that subject's body weight immediately before the administration of the compounds or compositions of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk for obesity. Another outcome of prevention may be decreasing the occurrence and/or severity of obesity-related disorders if the treatment is administered prior to the onset of obesity in a subject at risk for obesity. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

Obesity-related disorders are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia. Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compositions of the present invention are also useful for reducing the risk of secondary outcomes of obesity, such as reducing the risk of left ventricular hypertrophy. The compositions of the present invention are useful for treating patients with obesity-related disorders, as defined above.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes. Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compositions of the present invention are also useful for treating and/or preventing gestational diabetes mellitus.

As used herein, the term “substance abuse disorders” includes substance dependence or abuse with or without physiological dependence. The substances associated with these disorders are: alcohol, amphetamines (or amphetamine-like substances), caffeine, cannabis, cocaine, hallucinogens, inhalants, marijuana, nicotine, opioids, phencyclidine (or phencyclidine-like compounds), sedative-hypnotics or benzodiazepines, and other (or unknown) substances and combinations of all of the above.

In particular, the term “substance abuse disorders” includes drug withdrawal disorders such as alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances. It will be appreciated that reference to treatment of nicotine withdrawal includes the treatment of symptoms associated with smoking cessation.

Other “substance abuse disorders” include substance-induced anxiety disorder with onset during withdrawal; substance-induced mood disorder with onset during withdrawal; and substance-induced sleep disorder with onset during withdrawal.

For the treatment of substance abuse disorders, it may be useful to include in the compositions of the present invention a nicotinic receptor partial agonist such as varenicline or SR 591813; or an antidepressant such as bupropion, doxepine, or nortriptyline; or an anxiolytic agent such as buspirone or clonidine.

Representative experimental procedures are provided below. These are exemplary only and should not be construed as being limitations on the novel compositions and processes of this invention.

Abbreviations: DMF: dimethylformamide; ee: enantiomeric excess; HLB: hydrophilic-lipophilic balance; in: inches; LCAP: liquid chromatography assay percent; LCT: long chain triglyceride; MCT: medium chain triglyceride; Me: methyl; MTBE: methyl tert-butyl ether; PG: propylene glycol; RT: room temperature; SOLKA FLOC: filter aid.

### PREPARATORY EXAMPLE 1

### N-[1S,2S]-3-(4-chlorophenyl)-2-(3-cyanophenyl)-1-methylpropyl]-2-methyl-2-{[5-(trifluoromethyl pyridin-2-yl)oxy]propanamide MTBE hemisolvate

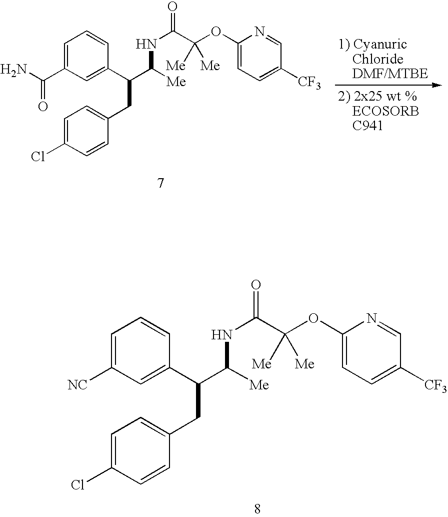

A solution of 470 g of 3-{(1S,2S)-1-(4-chlorobenzyl)-2-[(2-methyl-2-{[5-(trifluoromethyl)pyridine-2-yl]oxy}propanoyl)amino]-propyl}benzamide in DMF is transferred to a 12 L 4-necked round bottom flask equipped with mechanical stirrer, thermocouple, and 2 L addition funnel. Cyanuric chloride (103 g) is slurried in 2 L of MTBE and the resulting slurry was charged to the reaction via the 2 L addition funnel over ˜10 minutes. The reaction mixture is aged with stirring for 1 hour. The batch is cooled to 10° C. and diluted with 3 L of MTBE. 2 L of water and 2 L of saturated NaHCO₃ solution are added to the reaction while keeping the temperature below 20° C. The resulting slurry is transferred to a 50 L extractor containing 3 L of MTBE, 3 L of water, and 3 L of sat'd NaHCO₃. An additional 12 L of water is added to the batch and the layers are allowed to settle. The organic layer is washed twice with 3 L of water.

Ecosorb Treatment/Hemisolvate Isolation: The organic layer is azeotroped at 35° C., 17 in Hg to bring the KF to 219 (spec. at 500) while maintaining a volume of ˜11 L. The batch is then treated with 320 g of ECOSORB C941. The batch is aged for 4 hours at 50° C., then filtered over a pad of SOLKA FLOC and washed with 6 L of MTBE. The resulting filtrate is recharged to a 22 L vessel, concentrated to 11 L volume, and retreated with 116 g of ECOSORB C941. This slurry is filtered over a bed of SOLKA FLOC, and washed with 6 L MTBE. The resulting colorless MTBE layer is transferred through a 1 micron inline filter into a 12 L, 4 neck round bottom flask equipped with overhead stirrer and thermocouple, and concentrated to 2 L volume at 17 in Hg, 35° C. The batch is cooled to RT, and a sample is removed to create a seed bed. Once the sample is crystallized, it is returned to the flask, and the batch is aged for 30 minutes, creating a large seed bed. The isolated solid is dried over a stream of nitrogen to afford the title compound as an MTBE hemisolvate.

### PREPARATORY EXAMPLE 2

### Isolation of N-[1S,28]-3-(4-chlorophenyl)-2-(3-cyanophenyl)-1-methylpropyl]-2-methyl-2-{[5-(trifluoromethylpyridin-2-yl oxy]propanamide Polymorph B

In a 3 L, 3 neck round bottom flask equipped with overhead stirrer and thermocouple, 350 g of N-[1S,2S]-3-(4-chlorophenyl)-2-(3-cyanophenyl)-1-methylpropyl]-2-methyl-2-{[5-(trifluoromethyl pyridin-2-yl)oxy]propanamide hemisolvate was slurried in a total of 1.82 L of 2:3 isopropyl acetate:heptane. The mixture was aged for 1 h, and then filtered over a very small bed of SOLKA FLOC, thoroughly pull the liquors from the filter bed to minimize the loss of mother liquors. The filter cake was washed with 1 L of 1:3 IPAc: heptane into a separate flask. The two filtrates were combined (combined ee-98.5% ee). These two solutions were transferred by vacuum through a 1 micron inline filter into a 22 L 4 neck round bottom flask. The batch was heated to 45° C. over a steam pot, and then charged with 2.35 L of heptane. Seed of N-[1S,2S]-3-(4-chlorophenyl)-2-(3-cyanophenyl)-1-methylpropyl]-2-methyl-2-{[5-(trifluoromethylpyridin-2-yl)oxy]propanamide Polymorph B (Polymorph B seed was obtained from the same solvent system over a long time frame) (15.0 g) was added and the batch was aged at 45° C. overnight. The resulting slurry was then charged with 150 mL of heptane over 5 hours, then 220 mL heptane at 2.0 mL/min, then 1131 mL of heptane at 9 mL/min, then 6783 mL of heptane at 60 mL/min. Once all heptane was charged, the batch was cooled to RT and aged overnight. The batch was cooled to 0° C. and aged for 1 hour, filtered, and washed with 1 L of heptane to afford the title compound, crystal Form B (287 g, 87% isolated yield (from hemisolvate and corrected for seed), 98.6% ee, 99.5 LCAP, 99.5 wt % assay).

### EXAMPLE 1

### Solubility of N-[1S,2S]-3-(4-chlorophenyl)-2-(3-cyanophenyl)-1-methylpropyl]-2-methyl-2-{[5-trifluoromethyl]pyridine-2-yl}oxy}propanamide anhydrous, unsolvated Polymorph B in Various Liquid Vehicles

Solubility determinations were carried out at room temperature unless otherwise specified. Solubility of N-[1S,2S]-3-(4-chlorophenyl)-2-(3-cyanophenyl)-1-methylpropyl]-2-methyl-2-{[5-trifluoromethyl]pyridine-2-yl}oxy}propanamide (Compound I) as anhydrous unsolvated Polymorph B (such as prepared in Preparatory Example 2) was determined by preparing a suspension of anhydrous unsovated Polymorph B of Compound I in the solvent system. After equilibration for at least 24 hours, the suspension was filtered and the supernatant was analyzed by HPLC. Chromatography was performed on either a Vydac C₁₈ 300 A 250×4.6 mm 5 um particle size with in-line Phenomenex Security Guard w/ C₁₈ cartridge or on a Polaris C₁₈-Ether columns or on a Polaris C₈-Ether columns using 0.1% phosphoric acid in combination with methanol or acetonitrile depending on HPLC conditions. The compound was detected at 220 and 272 m wavelength and assayed using standard curves. The results are presented below in Table 1, calculated based on mg/g vehicle.

TABLE 1

| | Solubility of |
|---|---|
| | Compound I, mg/g of |
| Solvent | vehicle |
| CAPRYOL PGMC | 396 |
| IMWITOR 742 | 267 (at 30 C.) |
| MIGLYOL 812-IMWITOR 742, 1:1 v/v | ≧204 |
| IMWITOR 742-TWEEN 80, (70:30), w/w | 222 |
| IMWITOR 742-TWEEN 80 (60:40), w/w | 209 |
| LABRASOL | 202 |
| IMWITOR 742-MIGLYOL 812-TWEEN | ≧191 |
| 80,(45:45:10) w/w/w | |
| IMWITOR 742-MIGLYOL 812-TWEEN 80, | 187 |
| (40:40:20) w/w/w | |
| IMWITOR 742-TWEEN 80 (50:50) w/w | 184 |
| IMWITOR 742-TWEEN 80-Oleic Acid (5:3:2), | 178 |
| w/w/w | |
| MIGLYOL 812-IMWITOR 742, 4:1 v/v | 173 |
| IMWITOR 742-MIGLYOL 812-TWEEN 80, | 171 |
| (30:50:20) w/w/w | |
| IMWITOR 742-TWEEN 80-Oleic Acid (4:2:4), | 159 |
| w/w/w | |
| MIGLYOL 812-LABRASOL/TWEEN80/Oleic | 158 |
| acid (40:25:25:10), w/w/w/w | |
| IMWITOR 742-MIGLYOL 812-TWEEN 80, | 140 |
| (15:65:20) w/w/w | |
| IMWITOR 742-Oleic Acid-TWEEN 80- | 140 |
| MIGLYOL 812 (2:4:2:2), w/w/w/w | |
| PEG400-TWEEN80 (1:1), w/w | 138 |
| CREMOPHOR-EL | 131 |
| MIGLYOL 812/Oleic acid/CREMOPHOR EL | 129 |
| (5:2:3), w/w/w/w | |
| TWEEN 80-LABRASOL-Oleic Acid (25:25:50), | 120 |
| w/w/w | |
| PEG400 | 120 |
| MIGLYOL 812/Oleic acid/TWEEN 80 (5:2:3), | 120 |
| w/w/w/w | |
| MIGLYOL 812-PECEOL, 1:1 v/v | 118 |
| MIGLYOL 812-TWEEN 80, 1:1 w/w | 113 |
| TWEEN 80 | 104 |
| MIGLYOL 812-Corn oil-SPAN 80-TWEEN 80 | 97 |
| (30:20:25:25), w/w/w | |
| MIGLYOL 812 | 94 |
| LABRAFIL M1944CS | 84 |
| Corn oil-SPAN 80-TWEEN 80 | 78 |
| (30:30:40), w/w/w | |
| Corn oil-SPAN 80-CREMOPHOR EL | 69 |
| (35:35:30), w/w/w | |
| Soybean oil-SPAN 80-TWEEN 80 (50:25:25), | 59 |
| w/w/w | |
| Corn oil/Oleic acid/TWEEN 80 (5:2:3), w/w/w | 57 |
| SPAN 80 | 45 |
| PG | 28 |
| Soybean Oil | 21 |
| Oleic Acid | 20 |
| Olive oil | 18 |
| Glycerol | 0.4 |

### EXAMPLE 2

An example of the procedure used to prepare capsule dosage forms for Compound I is given below:

1. The mono- and diglycerides excipient (e.g., IMWITOR 742) is melted at an appropriate temperature.

2. Polysorbate 80 is added and mixed with the mono- and diglycerides at an appropriate temperature.

3. The Compound I is added to the mixture and dissolved.

4. The mixture is filled into hard gelatin capsules or suitably formulated soft gelatin capsules. For hard gelatin capsules, the filled capsules are sealed appropriately.

### EXAMPLE 3

### Mean Pharmacokinetic Parameters after Oral Administration of 50 mg Compound I in Liquid-Filled Gelatin Capsules to Male Rhesus Monkeys (Mean +/−SD)

Fasted male Rhesus monkeys (New Iberia, La.) were used for the monkey studies. All animals were fasted for 16 hours prior to dosing. They were housed in an AAALAC-accredited facility in accordance with USDA guidelines. After an overnight fast, capsules were administered to the monkeys orally via gavage tube and were followed immediately by 20 mL of water. Each formulation was tested in three monkeys (n=3). Water was returned at 1 hour after dosing and food was returned at 4 hours after dosing. Blood was drawn via venipuncture using a 21 g butterfly needle inserted into the saphenous vein at pre-dose and 15, 30, 60, 120, 240, 360, 480, and 1440 minutes after dosing. The plasma was separated by centrifugation (15 minutes at 2500 rpm) and kept frozen at −70° C. until analysis by LC/MS/MS.

A sensitive analytical method using liquid chromatography/electrospray ionization tandem mass spectrometry (LC/ESI-MS/MS) for the quantitation of Compound I in monkey plasma was developed and validated. The method employed a protein precipitation procedure using acetonitrile to isolate Compound I from the biological matrix. An analog of Compound I, N-[3-(4-fluoro-phenyl)-2-(3-cyano-phenyl)-1-methylpropyl]-2-(5-trifluoromethyl-2-pyridyloxy)-2-methylpropanamide; was used as the internal standard. Reconstituted extracts were ionized by a TurboIonSpray interface and analyzed in the selected reaction monitoring (SRM) mode. Chromatography was performed on a 100×2 mm, 5 μm, AQUASIL C8 column using 75:25 acetonitrile and 25 mM ammonium formate, pH 3.0. Under these conditions, no interference was observed for either Compound I or the internal standard from the endogenous components of dog plasma. The assay had a lower limit of quantitation (LOQ) of 1 ng/mL in plasma for Compound I based on 0.1-mL aliquots of plasma. The standard curve range was from 1 to 5000 ng/mL. The analysis time was 5.0 minutes per sample.

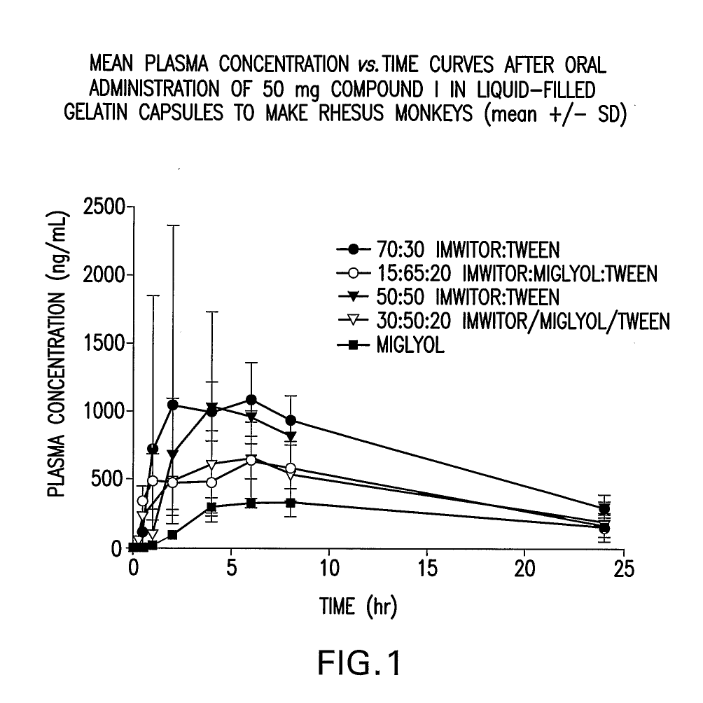

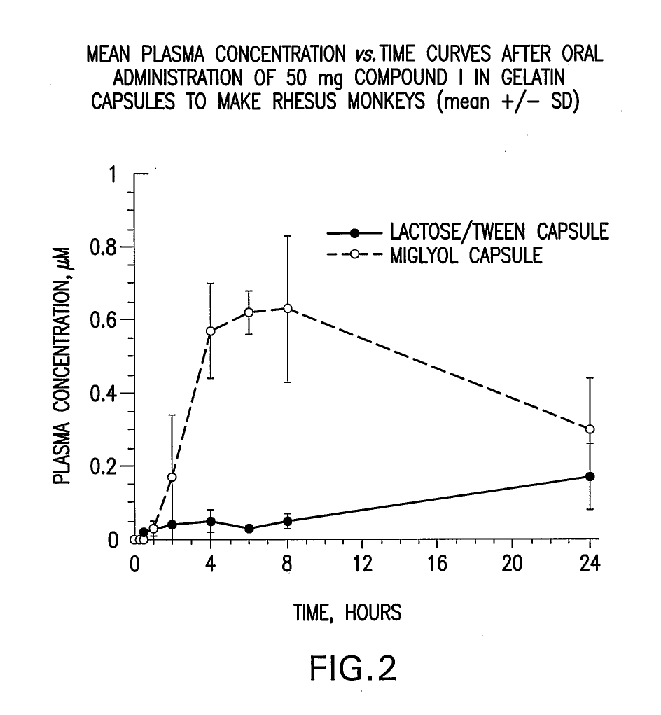

Area under the curve (AUC₀₋₂₄), mean and standard deviation of AUC, observed maximum plasma concentration (Cₘₐₓ), and time of Cₘₐₓ (Tₘₐₓ) were calculated with WinNonLin v3.1. The data are shown below in Table 2, as well as in FIGS. 1 and 2.

TABLE 2

| | AUC₀₋₂₄ | Cₘₐₓ | Tₘₐₓ |
|---|---|---|---|
| | (μM · hr) | (μM) | (hr) |
| IMWITOR 742/TWEEN | 33.01 ± 12.51 | 2.88 ± 1.83 | 4.67 ± 2.31 |
| 80 (70:30) | | | |
| IMWITOR 742/MIGLYOL | 19.31 ± 4.20 | 1.45 ± 0.28 | 6.00 ± 2.00 |
| 812/TWEEN 80 | | | |
| (15:65:20) | | | |
| IMWITOR 742/TWEEN | 28.93 ± 3.53 | 2.11 ± 0.20 | 4.67 ± 1.15 |
| 80 (50:50) | | | |
| IMWITOR 742/MIGLYOL | 19.69 ± 6.40 | 1.41 ± 0.31 | 4.00 ± 2.00 |
| 812/TWEEN 80 | | | |
| (30:50:20) | | | |
| Lactose/TWEEN 80 | 2.08 ± 0.83 | 0.17 ± 0.09 | 24.00 ± 0.00 |
| MIGLYOL 812 | 10.74 ± 1.99 | 0.73 ± 0.04 | 6.67 ± 2.31 |

### EXAMPLE 4

### Formulation of Soft-Gel Capsules

| | Dose | | |
|---|---|---|---|
| Composition (mg/unit dose) | 2 mg | 4 mg | 6 mg |
| Fill | | | |
| Compound I | 2.000 | 4.000 | 6.000 |
| Polysorbate 80 | 124.0 | 123.0 | 122.0 |
| (CRILLET 4HP) | | | |
| Medium Chain Partial | 124.0 | 123.0 | 122.0 |
| Glycerides (IMWITOR 742) | | | |
| Total Fill | 250.0 | 250.0 | 250.0 |
| Capsule Shell¹ | 152.0 | 152.0 | 152.0 |
| Capsule Weight (mg)² | 402.0 | 402.0 | 402.0 |
| Shape | Oval | Oval | Oval |
| Size | | | |
| Major axis (mm) | 13.63 ± 0.11 | 13.63 ± 0.11 | 13.63 ± 0.11 |
| Minor axis (mm) | 7.18 ± 0.10 | 7.18 ± 0.10 | 7.18 ± 0.10 |

¹This is a typical shell weight.
²This is a typical softgel weight. While the fill weight is well controlled, the shell weight may vary leading to a range of acceptable softgel weights.

Manufacturing Process (Fill Compounding):

Compound I was added to the IMWITOR 742, and the contents were mixed at 40+/−5° C. until Compound I was dissolved. Polysorbate 80 was added to the mixture of Compound I and IMWITOR 742. The solution was well mixed at 40+/−5° C. The solution was filtered through a 35 micron mesh filter. The solution was deaerated under vacuum until visual examination revealed that all air was removed (at least one hour).

The fill mixture and the gelatin mixture were compounded separately. These materials were then fed into the encapsulation machine. To encapsulate the fill solution, the gelatin formulation was cast into sheets on two cooled rollers. These sheets were passed through a series of rolls where a food grade lubricant was applied. The sheets were then fed through the rotary die rolls where the softgel was formed. As the lower edge of the softgel was formed, a reciprocating pump injected the fill solution into the center of the softgel after which the upper edge of the die came together to seal the softgel. The newly formed softgels were dislodged from the sheet and pneumatically conveyed to a tumble dryer where they stayed for 45-60 minutes. Upon exiting the dryer, the softgels were spread on trays and placed in a drying tunnel (low humidity chamber) and dried. Upon completion of the drying process, the softgels were visually inspected for defects. Subsequently, the capsules were sized to remove oversize and undersized capsules and polished.

### EXAMPLE 5

### Formulations of Opaque Hard Gelatin, Liquid Filled Capsules

| | Dose | | | |
|---|---|---|---|---|
| Composition (kg/batch) | 0.1 mg | 0.5 mg | 2 mg | 5 mg |
| Fill | | | | |
| Compound I | 8.6 × 10⁻³ | 43 × 10⁻³ | 0.172 | 0.570 |
| Polysorbate 80 (CRILLET | 17.2 | 17.178 | 17.144 | 22.516 |
| 4HP) | | | | |
| Medium Chain Partial | 17.2 | 17.178 | 17.144 | 22.516 |
| Glycerides (IMWITOR 742) | | | | |
| Total Fill (kg/batch) | 34.4 | 34.4 | 34.4 | 45.6 |
| Capsule Shell^{1,} H. G. Licap | 77.1 | 77.1 | 77.1 | 77.1 |
| Opaque White (mg/unit) | | | | |
| Batch size (theoretical) | 86,000 | 86,000 | 86,000 | 114,000 |
| Capsule Weight (mg/unit) | 477.1 | 477.1 | 477.1 | 477.1 |

¹This is a typical shell weight.

The IMWITOR 742 was melted at 40+/−5° C. Polysorbate 80 was added to an appropriately sized jacketed vessel and mixing was initiated. IMWITOR 742 was added to the Polysorbate 80 and the solution was mixed at 40+/−5° C. to obtain homogeneity. Compound I was slowly added to the mixture and dissolved. In process samples were taken after at least 1 hr of mixing and they were visually inspected for the presence of particulates and analyzed by HPLC to verify that the solution concentration reached the target value. The solution was filtered through a 100 mesh screen using a peristaltic pump into a receiving vessel. Using a peristaltic pump the solution was pumped to a ZANASI 40E hopper for encapsulation. The liquid formulation was dispensed into the size 1, white, opaque hard gelatin capsules (CAPSUGEL, containing gelatin and titanium dioxide) to a target fill weight of 400 mg. The filled capsules were transferred to a LEMS 30 capsule sealer and they are sealed by spraying with a mixture of 1:1 (weight:weight) water:ethanol (dehydrated, 190 proof) solution. After spraying the capsules were dried by gentle heating to approximately 45° C. The sealed capsules were placed onto trays lined with tray paper and were placed into a depression chamber (ZANASI 40E vacuum trap). After the completion of the vacuum cycle the capsules were visually inspected for leaking. The acceptable capsules are passed through a ZANASI capsule sorter to remove empty capsules. The finished capsules were then packaged into appropriate containers.

### EXAMPLE 6

### Formulations of Soft-Gel Capsules

| | Dose | | |
|---|---|---|---|
| Composition (mg/unit dose) | 2 mg | 4 mg | 6 mg |
| Fill | | | |
| Compound I | 2.000 | 4.000 | 6.000 |
| Polysorbate 80 | 123.9 | 122.9 | 121.9 |
| (CRILLET 4HP) | | | |
| Medium Chain Partial | 123.9 | 122.9 | 121.9 |
| Glycerides (IMWITOR 742) | | | |
| Butylated Hydroxyanisole | 0.1500 | 0.1500 | 0.1500 |
| (TENOX BHA flakes) | | | |
| Total Fill | 250.0 | 250.0 | 250.0 |
| Capsule Shell¹ | 152.0 | 152.0 | 152.0 |
| Capsule Weight (mg)² | 402.0 | 402.0 | 402.0 |
| Shape | Oval | Oval | Oval |
| Size | | | |
| Major axis (mm) | 13.63 ± 0.11 | 13.63 ± 0.11 | 13.63 ± 0.11 |
| Minor axis (mm) | 7.18 ± 0.10 | 7.18 ± 0.10 | 7.18 ± 0.10 |

¹This is a typical shell weight.
²This is a typical softgel weight. While the fill weight is well controlled, the shell weight may vary leading to a range of acceptable softgel weights.
Manufacturing process (fill compounding):
1. BHA is added to IMWITOR 742 and heated to 40 +/− 5° C.
2. Compound I is added to the IMWITOR 742/BHA, and the contents are mixed at 40 +/− 5° C. until the Compound I is dissolved.
3. Polysorbate 80 is added to the mixture of Compound I, IMWITOR 742/BHA. The solution is well mixed at 40 +/− 5° C.
4. The solution is filtered through a 35 micron mesh filter.
5. The solution is deaerated under vacuum until visual examination reveals that all air is removed (at least one hour).

The fill mixture and the gelatin mixture are compounded separately. These materials are then fed into the encapsulation machine. To encapsulate the fill solution, the gelatin formulation is cast into sheets on two cooled rollers. These sheets are passed through a series of rolls where a food grade lubricant is applied. The sheets are then fed through the rotary die rolls where the softgel is formed. As the lower edge of the softgel is formed, a reciprocating pump injects the fill solution into the center of the softgel after which the upper edge of the die comes together to seal the softgel. The newly formed softgels are dislodged from the sheet and pneumatically conveyed to a tumble dryer where they stay for 45-60 minutes. Upon exiting the dryer, the softgels are spread on trays and placed in a drying tunnel (low humidity chamber) and dried.

While the invention has been described and illustrated with reference to certain particular embodiments thereof, those skilled in the art will appreciate that various changes, modifications and substitutions can be made therein without departing from the spirit and scope of the invention. For example, solvents other than the particular solvents as set forth herein above may be useful in the chemical syntheses described herein. It is intended, therefore, that the invention be defined by the scope of the claims which follow and that such claims be interpreted as broadly as is reasonable.

1. A composition comprising N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or a pharmaceutically acceptable salt or solvate thereof, and a lipophilic vehicle selected from digestible oils, surfactants, cosolvents, and mixtures of any two or more thereof.

2. A composition comprising: N-[1S,2S]-3-[(4-chlorophenyl)-2-(3-cyanophenyl)-1-methylpropyl]-2-methyl-2-{[(5-trifluoromethyl)pyridine-2-yl]oxy}propanamide, or a pharmaceutically acceptable salt or solvate thereof, and a pharmaceutically acceptable carrier selected from: a high HLB surfactant, a low HLB surfactant, a digestible oil, a co-solvent, and mixtures of any two or more thereof.

3. The composition according to claim 2 wherein: the high HLB surfactant has an HLB of from 8 to 20; the low HLB surfactant has an HLB of less than 8; the digestible oil is selected from: vegetable oils, medium chain triglycerides, long chain triglycerides, mixtures of mono-, di- and tri-glycerides, and lipophilic derivatives of fatty acids; and the cosolvent is selected from: polyol esters of fatty acids, triacetin, diethylene glycol monoethyl ether, glycofurol, peppermint oil, 1,2-propylene glycol, ethanol, oleic acid, and other higher and lower molecular weight polyethylene glycols.

4. The composition according to claim 3, wherein:
the high HLB surfactant is selected from: Polysorbate 80, TWEEN 80, CRILLET 4 HP, CRILLET 4NF, TWEEN 20, CRILLET 1, CREMOPHOR RH40, CREMOPHOR RH60, CREMOPHOR EL, ETOCAS 30, NIKKOL HCO-60, Vitamin E TPGS, LABRASOL, ACCONON MC-8, GELUCIRE 50/13, GELUCIPE 44/14, MYRJ, BRIJ, and POLOXAMERS;
the low HLB surfactant is selected from: CAPMUL MCM, CAPMUL MCM 8, CAPMUL MCM 10, IMWITOR 988, IMWITOR 742, IMWITOR 308, LABRAFIL M 1944 CS, LABRAFIL M 2125, CAPRYOL PGMC, CAPRYOL 90, LAUROGLYCOL, CAPTEX 200, MIGLYOL 840, PLUROL OLEIQUE, SPAN 80, SPAN 20, CRILL 1, CRILL 4, MAISINE, and PECEOL;
the digestible oil is selected from: MIGLYOL 812, MIGLYOL 810, NEOBEE MS, CAPTEX 300, CAPTEX 355, LABRAFAC CC, CRODAMOL GTCC, soybean oil, safflower oil, corn oil, olive oil, cottonseed oil, arachis oil, sunflowerseed oil, palm oil, rapeseed oil, ethyl oleate, and glyceryl monooleate; and semisolid vehicles such as IMWITOR 491, IMWITOR 900, GELUCIRE 33/01, GELUCIRE 39/01, GELUCIRE 43/01, and SOFTISANS, and the cosolvent is as in claim 3.

5. The composition according to claim 2, wherein the pharmaceutically acceptable carrier comprises the high HLB surfactant Polysorbate 80 and the low HLB surfactant mono- and di-glycerides.

6. The composition according to claim 5 additionally comprising an antioxidant.

7. A capsule comprising about 0.1 mg to about 10 mg N-[1S,2S]-3-(4-chlorophenyl)-2-(3-cyanophenyl)-1-methylpropyl]-2-methyl-2-{[5-trifluoromethyl]pyridine-2-yl}oxy}propanamide or a pharmaceutically acceptable salt or solvate thereof, and a pharmaceutically acceptable carrier selected from: a high HLB surfactant, a low HLB surfactant, a digestible oil, and a cosolvent.

8. The capsule according to claim 7, wherein the high HLB surfactant
is selected from: Polysorbate 80, TWEEN 20, CREMOPHOR RH40, CREMOPHOR RH60, CREMOPHOR EL, ETOCAS 30, NIKKOL HCO-60, Vitamin E TPGS, LABRASOL, ACCONON MC-8, GELUCIRE 44/14, MYRJ, BRIJ;
the low HLB surfactant is selected from: CAPMUL MCM, CAPMUL MCM 8, CAPMUL MCM 10, IMWITOR 988, IMWITOR 742, IMWITOR 308, LABRAFIL M 1944 CS, LABRAFIL M 2125, LAUROGLYCOL, CAPTEX 200, MIGLYOL 840, PLUROL OLEIQUE, SPAN 80, SPAN 20, CRILL 1, CRILL 4, CAPRYOL PGMC, MAISINE, and PECEOL;
the digestible oil is selected from: MIGLYOL 812, MIGLYOL 810, NEOBEE MS, CAPTEX 300, CAPTEX 355, CRODAMOL GTCC, soybean oil, safflower oil, corn oil, olive oil, cottonseed oil, arachis oil, sunflowerseed oil, palm oil, rapeseed oil, ethyl oleate, and glyceryl monooleate; and
the cosolvent selected from: polyol esters of fatty acids, trialkyl citrate esters, propylene carbonate, dimethylisosorbide, ethyl lactate, N-methylpyrrolidones, transcutol, glycofurol, peppermint oil, 1,2-propylene glycol, ethanol, oleic acid, PEG 400 and other higher and lower molecular weight PEGs, and polyethylene glycol.

9. The capsule according to claim 7, wherein the pharmaceutically acceptable carrier comprises the high HLB surfactant Polysorbate 80 and the low HLB surfactant mono- and di-glycerides.

10. The capsule according to claim 7, wherein the amount of N-[1S,2S]-3-(4-chlorophenyl)-2-(3-cyanophenyl)-1-methylpropyl]-2-methyl-2-{[5-trifluoromethyl]pyridine-2-yl}oxy}propanamide is selected from: 0.1 mg, 0.5 mg, 1 mg, 2 mg, 2.5 mg, 3 mg, 4 mg, 5 mg, 6 mg, 7 mg, 7.5 mg, 8 mg, 9 mg, and 10 mg.

11. The capsule according to claim 9, wherein the Polysorbate 80 is selected from TWEEN 80, CRILLET 4 HP, and CRILLET 4 NF; the mono- and di-glycerides is IMWITOR 742, and the Polysorbate 80 and the mono- and diglycerides are present in equal amounts by weight.

12. The capsule according to claim 11 additionally comprising an antioxidant.

13. The capsule according to claim 10, wherein the capsule is selected from a soft-gelatin capsule and a hard gelatin capsule.

14. The capsule according to claim 12, wherein the capsule is a soft-gelatin capsule.

15. A process for preparing the capsule of claim 7, comprising the steps of:
(a) blending the carrier ingredients selected from: a high HLB surfactant, a low HLB surfactant, a digestible oil, and a cosolvent to form a vehicle;
(b) dissolving of N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or a pharmaceutically acceptable salt or solvate thereof in the vehicle to form a solution or dispersion; and
(c) encapsulating the solution or dispersion of N-[1S,2S]-3-(4-chlorophenyl)-2-(3-cyanophenyl)-1-methylpropyl]-2-methyl-2-{[5-trifluoromethyl]pyridine-2-yl}oxy}propanamide or a pharmaceutically acceptable salt or solvate thereof in the vehicle in hard or soft gelatin capsules.

16. A process for preparing the capsule of claim 9, comprising the steps of:
(a) heating the low HLB surfactant;
(b) adding N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or a pharmaceutically acceptable salt or solvate thereof, to the heated low HLB surfactant and maintaining the heated temperature until the N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or pharmaceutically acceptable salt or solvate thereof, is dissolved;
(c) adding the high HLB surfactant to the solution of N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or the pharmaceutically acceptable salt or solvate thereof, and stirring the resulting mixture; and
(d) encapsulating the mixture of N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or the pharmaceutically acceptable salt or solvate thereof, in appropriate hard or soft gelatin capsules.

17. A process for preparing a capsule according to claim 12, wherein the antioxidant is butylated hydroxyanisole, the low HLB surfactant is IMWITOR 742 and the high HLB surfactant is Polysorbate 80, comprising the steps of:
(a) adding butylated hydroxyanisole to the IMWITOR 742, and heating to 40+/−5° C.;
(b) adding N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or a pharmaceutically acceptable salt or solvate thereof, to the IMWITOR 742/butylated hydroxyanisole, and mixing at 40+/−5° C. until the N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or the pharmaceutically acceptable salt or solvate thereof is dissolved;
(c) adding Polysorbate 80 to the solution of N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or the pharmaceutically acceptable salt or solvate thereof, in IMWITOR 742/butylated hydroxyanisole and mixing; and
(d) encapsulating the mixture of N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or the pharmaceutically acceptable salt or solvate thereof in the appropriate hard or soft gelatin capsules.

18. The composition according to claim 2, which comprises the following amounts of components, by weight percent: 0.01-25% N-[1S,2S]-3-[(4-chlorophenyl)-2-(3-cyanophenyl)-1-methylpropyl]-2-methyl-2-{[(5-trifluoromethyl]pyridine-2-yl)oxy]propanamide or a pharmaceutically acceptable salt or solvate thereof, 0-70% digestible oil; 0-50% high HLB surfactant; and 0-70% low HLB surfactant.

19. The composition according to claim 2, which comprises the following amounts of components, by weight percent: 0.01-13% N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or a pharmaceutically acceptable salt or solvate thereof; 20-50% high HLB surfactant; and 40-80% low HLB surfactant.

20. The composition according to claim 6, which comprises the following amounts of components, by weight percent: 0.8% to 2.4% N-[1S,2S]-3-[(4-chlorophenyl)-2-(3-cyanophenyl)-1-methylpropyl]-2-methyl-2-{[(5-trifluoromethyl)pyridine-2-yl]oxy}propanamide or a pharmaceutically acceptable salt or solvate thereof, 48.7% to 49.6% Polysorbate 80, 48.7% to 49.6% IMWITOR 742, and 0.02-0.1% butylated hydroxyanisole.

21. The composition according to claim 1 which is an oral pharmaceutical composition.

22-23. (canceled)

24. A method of treating a condition selected from: diabetes, obesity and substance abuse disorders in a human in need of such treatment comprising administering the composition of claim 21.

25. A method of treating a condition selected from: diabetes, obesity and substance abuse disorders in a human in need of such treatment comprising administering the composition of claim 2.
